(19) United States
(12) Patent Application Publication
(10) Pub. No.: US 2008/0172130 A1
(43) Pub. Date: Jul. 17, 2008

(54) MODULAR ACETABULAR CUP AND ANCHORING SCREW FOR FIXING A PROSTHETIC IMPLANT

(76) Inventor: Frederique Macara, Carnon La Grande-Motte (FR)

Correspondence Address: EGBERT LAW OFFICES, 412 MAIN STREET, 7TH FLOOR, HOUSTON, TX, 77002, US

(21) Appl. No.: 12/037,052

(22) Filed: Feb. 25, 2008

Related U.S. Application Data
(63) Continuation-in-part of application No. 10/484,548, filed on Jan. 22, 2004, now abandoned.

(30) Foreign Application Priority Data
Jul. 23, 2003 (FR) 01/09796

(51) Int. Cl. A61F 2/32

(52) U.S. Cl. 623/22.21; 623/22.36

(57) ABSTRACT

A modular prosthetic acetabulum apparatus for use in reconstruction of a hip joint by placement in an acetabulum cavity has a supporting plate with a shape suitable for placement in the acetabulum cavity, an anchor cooperative with the supporting plate for adjustably positioning the supporting plate in the acetabulum cavity, a spacer locked onto the supporting plate and selectively rotatable with respect to the supporting plate, and a cup affixed within the spacer. The supporting plate has an annular internal surface cooperative and complementary to a tapered annular exterior surface of the spacer. The cup has a tapered exterior surface complementary to and cooperative with an annular tapered interior in the spacer.

### CROSS-REFERENCE TO RELATED APPLICATIONS

The present application is a continuation-in-part of U.S. application Ser. No. 10/484,548, filed on Jan. 22, 2005, and entitled “Modular Acetabular Cup, And Anchoring Screw For Fixing A Prosthetic Implant Such As An Acetabular Cup”, presently pending. U.S. application Ser. No. 10/484,548, claims priority from International Application No. PCT/FR02/02622, filed on Jul. 22, 2002 claiming priority from French application Ser. No. 10/09796 of Jul. 23, 2001.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

1. Field of the Invention

This invention relates to a modular prosthetic acetabulum for surgically treating the reconstruction of the hip joint in the case of total restoring of hip prostheses, with or without bone-material deficiency. The present invention further relates to an anchoring-screw device for fastening a prosthetic implant, such as the acetabulum. The present invention is intended to restore all of the biomechanical criteria of the joint by individually treating, through the modularity of the prosthetic implant, the basic elements relating to the fastening and orientation of the implant. As such, the modular acetabular prosthetic implant is adapted to the various needs that would arise for various patients while, at the same time, allowing for the efficient placement of the implant.

2. Description of Related Art Including Information Disclosed Under 37 CFR 1.97 and 37 CFR 1.98.

The current technique for acetabulum reconstruction shows a deficiency in the area of both the intra-ossa anchoring of the existing prosthetic devices and their bio-mechanical positioning. The anchoring with a limited primary fastening can indeed have, in the course of time, a harmful affect on the integration of the implant. This is the result of the compressive and twisting forces to which it is subjected. This can give rise to a loosening and/or a breaking of the installed material.

The positioning of the implant is often dependent on the state of the osseous acetabulum to be treated. The positioning of the implant does not allow a free orientation in all those planes which would allow the restoration of normal bio-mechanics of the joint. This can be prejudicial to the stability of the joint. It often causes dislocation and/or loosening of the implant.

These and other objects and advantages of the present invention will become apparent from a reading of the attached specification and appended claims.

### BRIEF SUMMARY OF THE INVENTION

The present invention is a modular prosthetic acetabulum apparatus for use in reconstruction of a hip joint by placement in an acetabulum cavity. The apparatus includes a supporting plate having a shape suitable for placement in the acetabulum cavity, an anchor cooperative with the supporting plate for adjustably positioning the supporting plate in the acetabulum cavity to a desired depth, a spacer locked onto the supporting plate and selectively rotatable with respect to the supporting plate, and a cup affixed within the spacer. The supporting plate has an annular interior surface which tapers so as to have a wide diameter at an end of the supporting plate and a narrow diameter spaced from supporting plate. The anchor is in the form of an anchoring screw. The spacer has an annular internal area. This annular tapered internal area of the spacer has a wide diameter at a first end of the spacer and a narrow diameter inwardly of the first end of the spacer. The spacer has an annular external area formed adjacent a second end of the spacer. This annular tapered external area has a narrow diameter adjacent the second end of the spacer and a wide diameter positioned inwardly of the second end of the spacer. The annular tapered external area of the spacer is juxtaposed against the tapered interior of the supporting plate such that the spacer is selectively rotatable within the supporting plate. The cup has a tapered exterior surface received in the tapered interior area of the spacer. The cup has an equatorial rim extending therearound and extending outwardly therefrom. The equatorial rim is positioned against the first end of the spacer.

The supporting plate has a plurality of external legs. At least one of the plurality of external legs has eyelets formed therein. Another of the plurality of external legs has an obturator hook extending therefrom. The fastening screw is of a spongy or cortical material received through one of the eyelets of the external leg. The supporting plate also has at least one internal leg. The internal leg has an eyelet formed therein. This eyelet has a frustoconical wall. The fastening screw is received through the eyelet and has a bearing surface with a hemispherical profile. The bearing surface bears against the frustoconical wall of the eyelet. The plurality of external legs of the supporting plate comprise a pair of external legs having eyelets formed therein. The plurality of external legs also comprises a single leg having the obturator hook formed thereon. The plurality of external legs are disposed at an acute angle with respect to each other on one side of the supporting plate. The single leg with the obturator hook extends from an opposite side of the supporting plate.

The tapered exterior surface of the cup is juxtaposed against the annular interior area of the spacer so as to be selectively rotatable within the annular interior area. The cup is, in the preferred embodiment, formed of a metallic material. Each of the supporting plate, the anchor, and the cup has an osteoconductive coating thereon.

The spacer has a locking collar extending outwardly from an exterior surface thereof. This locking collar extends around more that one-half of a circumference of the spacer. The locking collar has a plurality of eyelets formed in spaced relationship to each other along the locking collar. The supporting plate has a peripheral shoulder extending outwardly therefrom adjacent the end of the supporting plate. The peripheral shoulder has a plurality of tapped holes formed therein. At least one connecting screw is received in one of the plurality of eyelets and in one of the plurality of tapped holes so as to fix a rotation position of the spacer with respect to the supporting plate. Each of the plurality of tapped holes has a wall with a frustoconical shape. The connecting screw has a head with a shape bearing against the wall.

The cup has a smooth internal portion. A polymeric insert is received in the internal portion such that the polymeric insert moves freely therein. The insert has a hemispherical cavity suitable for receiving a head of a prosthetic femoral element. Alternatively, a ceramic insert is received in female portion of the cup. This insert has a hemispherical cavity for receiving a head of a prosthetic femoral element.

The anchoring means comprises a body having a cylindrically-shaped proximal portion and a sharp tip at a distal portion. The body has a spongy self-tapping outer thread. The proximal portion having a tapped channel formed axially therein. A threaded piston is received by the tapped channel. The threaded piston has a head and a tapped well. The tapped well is suitable for receiving a locking counter-screw therein. The counter-screw has a head with a curved hemispherically-shaped bearing surface. The eyelet has a profile complementary to the bearing surface.

The modular prosthetic acetabulum of the present invention presents a global solution for the surgical treatment of the hip joint reconstruction by taking into consideration of its mechanical, anatomical and biological requirements. This prosthetic acetabulum allows proper installation through the primary and secondary fastening means by way of the anchoring and fastening screws. The proper anatomical orientation of the implant is accomplished through its broad modularity. The stability is achieved by the Morse-tapers-type system for assembling each of the components together. The primary fastening of the prosthetic acetabulum is assured by the supporting plate through the use of anchoring screws. This allows an anchoring in a healthy osseous area by using all of the available supports without any orientation constraint. The multiplication of the anchoring and resting points of the supporting plate in the peripheral area are achieved through the supra-acetabular legs and the obturator hook and in the internal area through the ability of the acetabular legs to achieve a maximum stability of the supporting plate and, hence, of the implant.

In the case of bone destruction, the supra-acetabular fastening legs can also be fastened by means of anchoring screws identical to those being used for fastening the acetabular legs. The association of anchoring screws and support plate represents a true osteosynthesis. Its primary mechanical stability remains an essential element in the life expectancy of the reconstruction and allows for efficient loading into the patient.

The secondary fastening of the prosthetic acetabulum is optimized by filling the acetabular cavity with grafts and/or bone substitutes. This takes part in the reconstruction of the destroyed bone stock. A double osteoconductive coating can be applied primarily on the back of the acetabular leg or legs of the supporting plate as well as onto the back of the cup which is accommodated in the supporting plate through the spacer.

The orientation of the supporting plate, the spacer and the cup allows for a swiveling and/or rotation in all planes. This serves to act as an interface between the supporting plate and the cup and to allow one to center the anatomical resting point within the cup. This increases the congruency and the size of the bearing surface. The adjustment of the anatomical orientation of the prosthetic acetabulum is ensured by the cup. The metal cup is able to swivel over 180° in the vertical anteroposterior plane and by the swiveling spacer and the vertical and horizontal planes. As such, these components allow for the very fine adjustments of the anatomical axes of the joint.

The Morse-tapers-type assembling of the various components of the prosthetic acetabulum of the present invention ensures the locking of the entire end and makes long-term stability reliable.

The present invention, in which the insert is fixed to the cup, can serve to meet specific purposes, such as for very young or very active patients. This characteristics of the prosthetic acetabulum of the present invention guarantee the restoring of normal biomechanics and enhance the life expectancy of the prostheses. This allows for a faster rehabilitation and return to active life.

The anchoring screw is used in the present invention serves to fasten the prosthetic implant. The anchoring screw includes a suitable means for ensuring the fine adjustment of the height of the bearing surfaces of the implant. This allows for the anchoring of the implant in the bone tissue while ensuring a balanced adjustment of the compressive forces. The threaded piston associated with the fastening screw of the present invention allows, according to the depth of insertion into the tapped channel, the ability to adjust the height of the bearing surfaces of the prosthetic implant. The counter-screw ensures the tightening of the implant.

### BRIEF DESCRIPTION OF THE SEVERAL VIEWS OF THE DRAWINGS

FIG. 1 is a perspective view exploded view of a prosthetic acetabulum in accordance with the preferred embodiment of the present invention.

FIG. 2 is a cross-sectional view showing the assembly of the spacer and the cup within the supporting plate.

FIG. 3 is a detailed cross-sectional view of the circled area of FIG. 2.

FIG. 4 is a plan view of the assembled prosthetic acetabulum of the present invention.

FIG. 5 is a cross-sectional view of the assembled prosthetic acetabulum in accordance with an alternative embodiment of the present invention.

FIG. 6 is an isolated perspective view of the supporting plate of the prosthetic acetabulum of the present invention.

FIG. 7 is a cross-sectional view of an anchoring screw as used in the prosthetic acetabulum assembly of the present invention.

### DETAILED DESCRIPTION OF THE INVENTION

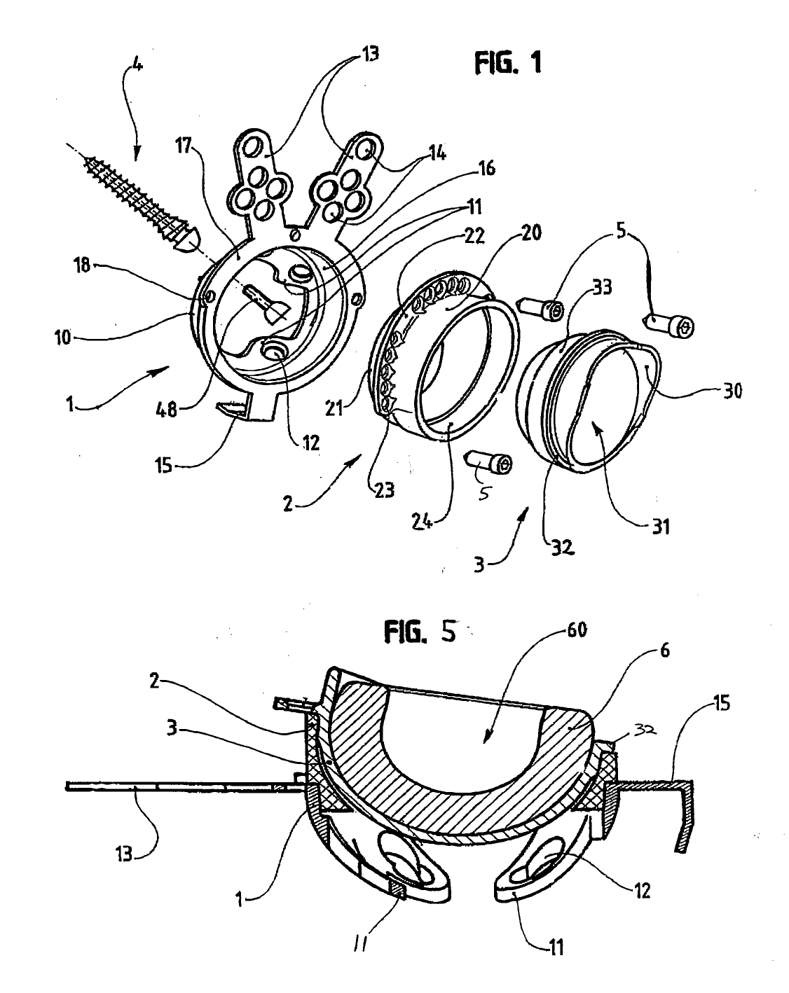

Referring to FIG. 1, there is shown the prosthetic acetabulum apparatus in accordance with the preferred embodiment of the present invention. The prosthetic acetabulum apparatus of the present invention includes a supporting plate 1, a spacer 2, a cup 3 and an anchoring screw 4. The spacer 2 has a distal area 21 received within a proximal area 16 on the supporting plate 1. The cup 3 has an annular external area 33 received with annular tapered interior area 24 of the spacer 2.

The supporting plate 1 is suitable for being implanted into the acetabular cavity. The supporting plate 1 has a collar 17 extending at one end thereof. The collar 17 has a plurality of tapped holes 18 formed therethrough. The proximal area 16 is a tapered annular area which has a wide diameter at the collar 17 at the end of the supporting plate 1 and a narrow diameter inwardly of the collar 17 of supporting plate 1. A plurality of legs 13 and 15 extend outwardly of the collar 17. The supra-acetabular legs 13 extend at an acute angle with respect to each other. A plurality of eyelets 14 are formed through the supra-acetabular legs 13. The leg 15 has an obturator hook formed inwardly therefrom. The leg 15 extends outwardly of the collar 17 on a side of the support plate 1 opposite the supra-acetabular legs 13. Internal legs 11 extend inwardly at the opposite end of the supporting plate 1 from the collar 17. Internal legs 11 have eyelets 12 formed therein. Each of the internal legs 11 has a lug formed thereon. A bearing ring 10 is formed at the end of the supporting plate 1 opposite the collar 17 and extends annularly therearound.

The spacer 2 has a ring 20 at a first end thereof and a distal area 21 at an opposite end thereof. A peripheral collar 22 extends around the spacer 2 between the ring 20 and the distal area 21. The distal area 21 is annular tapered external area. This annular tapered external area serves to fit complementarily within the tapered interior of the proximal area 16 of supporting plate 1. The peripheral collar 22 is configured so as to fit against the collar 17 during assembly. The peripheral collar 22 has a plurality of eyelets 23 formed therethrough. The peripheral collar 22 extends for at least more than one half the circumference of the spacer 2. The plurality of eyelets 23 are arranged in side-by-side relationship along the peripheral collar 22. As such, as the spacer 2 is rotated relative to the to the supporting plate 1, the eyelets 23 can align with the plurality of tapped holes 18 so as to allow for the fine adjustment of the spacer 2 with respect to the supporting plate 1. The ring 20 has an annular tapered exterior surface thereon. The annular tapered exterior surface of ring 20 tapers so as to have a wide diameter at the first end of the spacer 2 and a narrow diameter inwardly of this end of the spacer 2.

The cup 3 has a hemispherical cavity 31 formed therein. A surface 30 projects outwardly of the equatorial ring 32 of the cup 23. The equatorial ring 32 extends around more than one-half the circumference of the cup 3 between the projection 30 and the opposite end. The tapered external area 33 is formed so as to have a wide diameter adjacent to the equatorial ring 32 and a narrow diameter at the end of the cup 3. This tapered exterior area 33 is configured to fit within the tapered interior area 24 of the spacer 2. This arrangement allows fine adjustment between the cup 3 and the spacer 2 by simple rotation of the cup 3 with respect to spacer 2. As such, the arrangement of these tapered areas of the supporting plate 1, the spacer 2 and the cup 3 creates a Morse-tapers system whereby the proper adjustment of the components can be established.

The anchoring screw 4 has a particular configuration as illustrated in detail in FIG. 7. The anchoring screw 4 is configured so as to fix the supra-acetabular legs 13 to the bone material in the hip. The anchoring screw 4 can extend through the eyelets 14. Each of the eyelets 14 has a generally frustoconical wall. The head of the anchoring screw 4 is configured so as to be juxtaposed against frustoconical wall of the eyelets 14. The arrangement of the eyelets 14 allows the surgeon to properly choose an appropriate location for the anchoring of the supporting plate 1 onto the acetabulum area.

Fastening screws 5 are provided so as to join the spacer 2 in a desired location relative to the supporting plate 1. The fastening screws 5 will extend through at least some of the eyelets 23 on the peripheral collar 22 of spacer 2. The fastening screws 5 can then join with the supporting plate 1 by entering the select ones of the tapped holes 18. As can be seen, when the spacer 2 is properly rotated, at least some of the eyelets 23 will align with at least some of the tapped holes 18. The fastening screws 5 can then be placed through the eyelets 23 and joined to the tapped holes 18. Each of the eyelets 23 on the peripheral collar 22 of spacer 2 have a frustoconical wall. Each of the fastening screws 5 will have a head with a surface that will be juxtaposed against such frustoconical walls when the fastening screws are properly installed.

FIG. 2 illustrates the manner in which the tapered surface of each of supporting plate 1, the spacer 2 and the cup 3 are joined together so as to form the Morse-tapers system. The support plate 1 has an annular internal tapered area formed at the proximal area 16. As can be seen, the wide diameter of the tapered internal wall of the proximal area 16 is at one end of the supporting plate 1. The narrow diameter of the annular tapered internal area 16 is inwardly of this end of the support plate 1. The spacer 2 has its distal area 21 with a similarly tapered outer surface as the tapering of the internal area of the proximal area 16 of support plate 1. As can be seen, this annular externally tapered area 21 has a narrow diameter at one end of the spacer 2 and a wide diameter inwardly of this end of the spacer 2. As such, when the spacer 2 is fitted within the support plate 1, the tapered surfaces will be juxtaposed against each other. The spacer 2 can then be suitably rotated so as to achieve its proper angular orientation with respect to the support plate 1.

The support plate 1 is illustrated in FIG. 2 as having a tapered area 24 at an opposite end of the spacer 2. This tapered interior area 24 will have a wide diameter at the opposite end of the spacer 2 and a narrow diameter inwardly therefrom. The cup 3 has an annular tapered external area 33 formed along an outer surface thereof. This annular tapered external area 33 will have a narrow diameter spaced away from the equatorial rim 32 and a wide diameter adjacent to the equatorial rim 32. It can be see that the equatorial rim 32 will reside against the opposite end of the spacer 2 when the cup 3 is installed into the interior of the spacer 2. The tapered surfaces will allow a proper rotation of the cup 3 with respect to the spacer 2 so as to achieve further fine adjustment of the cup 3 with respect to the spacer 2.

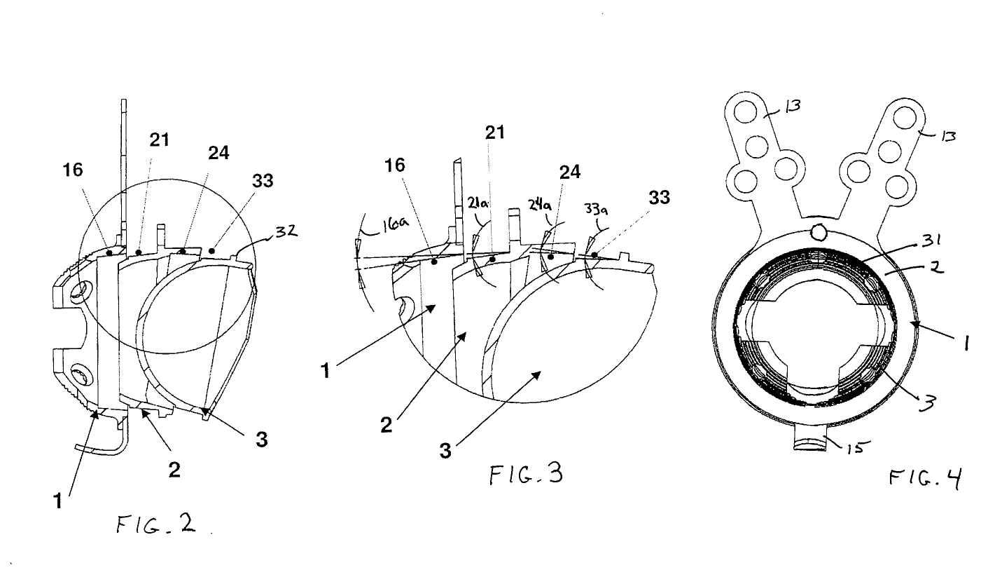

FIG. 3 shows, in greater detail, how the proximal area 16 of the support plate 1 is tapered by an angle 16a. The annular tapered external area at the distal area 21 of spacer 2 is tapered by an angle 21a. The annular tapered internal area 24 of spacer 2 is tapered by an angle 24a. The annular tapered external area 33 of the cup 3 is tapered by an angle 33a. In the present invention, the angle of taper 16a will be equal to the angle of taper 21a. Similarly, the angle of taper 24a will be equal to the angle of taper 33a.

FIG. 4 shows how the support plate 1 receives the spacer 2. FIG. 4 also shows that the cup 3 is received within the interior of the spacer 2. The cup 3 has a hemispherical cavity 31 formed in an interior thereof. The supra-acetabular legs 13 are illustrated as extending outwardly on one side of the support plate 1 at an acute angle with respect to each other. The leg 15 with the obturator hook extends outwardly on an opposite side of the support plate 1 from the supra-acetabular legs 13.

FIG. 5 shows an assembly of the components of the present invention. In FIG. 5, the support plate 1 has the supra-acetabular legs 13 extending outwardly therefrom. The leg 15 with the obturator hook extends outwardly on an opposite side of the support plate 1. The internal legs 11 are formed so as to extend inwardly at one end of the support plate 1. Eyelets 12 are formed through the internal legs 11 so as to allow for the receipt of anchoring screws 4 therein.

The spacer 2 is received within the proximal area 16 of the support plate 1. The cup 3 is received within the interior of the spacer 2. The equatorial rim 32 resides against the end of the spacer 2 so as to provide support therefore. Insert 6 is received within the hemispherical cavity 31 of the cup 3. The insert 6 can be of the nature of a polymeric insert which moves freely within the hemispherical cavity 31. This insert 36 has a hemispherical cavity 60 formed therein. Hemispherical cavity 60 is suitable for the receipt of a head of a prosthetic femoral element.

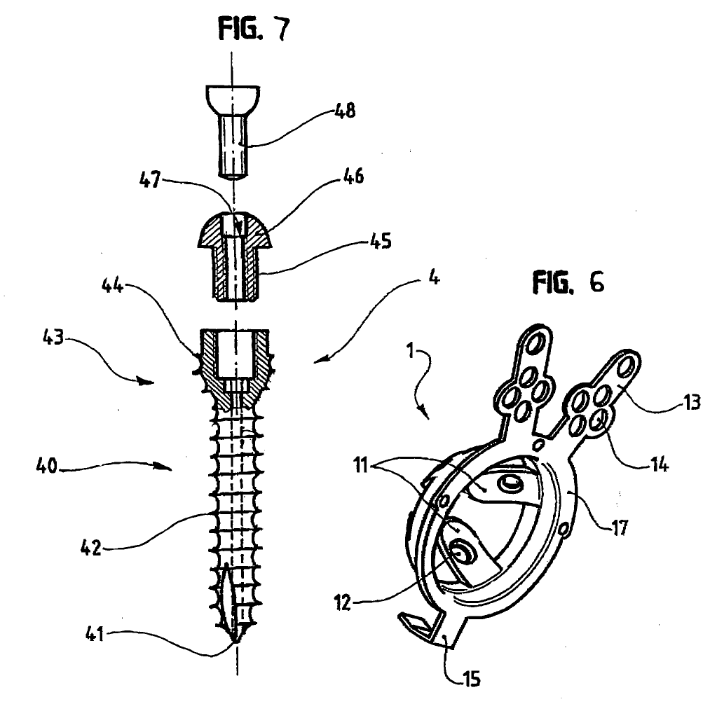

FIG. 6 is an isolated view of the support plate 1. As can be seen in FIG. 6, the support plate 1 has supra-acetabular legs 13 extending outwardly therefrom. Eyelets 14 are formed in the legs 13. The collar 17 extends around an end of the support plate 1. The internal legs 11 each have an eyelet formed therein. The leg 15 with the obturator hook is formed at an opposite side of the collar 17 from the supra-acetabular legs 13.

FIG. 7 illustrates the anchoring screw 4 which allows the surgeon to perform the adjustment of the positioning of the support plate 1 in the bone cavity. The anchoring screw 4 is preferably grooved in order to facilitate its installation. The anchoring screw 4 has a threaded distal portion 40 having a tip 41 and a spongy outer thread 42. The anchoring screw 4 has a proximal portion 43 having the shape of a cylinder with a tapped channel 44 extending in an axial direction thereof. A threaded piston 45 can be screwed into the tapped channel 44. The threaded piston 45 is provided with a head 46 and a tapped well 47 for receiving a locking counter-screw 43. The threaded piston 45 allows for the adjustment of the height to the bearing surfaces of the support plate 1. The counter screw 48 ensures the proper holding of the support plate 1. The threaded piston 45 can be adjustably inserted into the tapped channel 44. The various threads can have, preferably, reversed pitches.

After anchoring the distal portion 40 into the bone material, the screwing or unscrewing of the piston 45 allow for the adjustment of the height of the head 46. This serves as a support for the supporting plate 1 and, more particular, for the internal legs 11. After having determined the adequate length of insertion of the piston 45 in the channel 44, the supporting plate 1 is made integral by means of the locking counter-screw 48 as screwed into the well 47. The proximal portion 43, the piston 47 and the counter screw 48 are each provided with an operating die, such as an axial hollow die.

It should be noted that the fastening screws 5, the locking counter-screw 48, as well as the fastening screws (not shown) of the external supra-acetabular legs 13 have heads with a curved, hemispherical profile. This head has a bearing surface thereon. The various components 22, 34, 11 and 13, respectively, have eyelets 23, 35, 12 and 14 thereon. Each of these eyelets have a countersink with a profile complementary to the head of the various screws. As such, the orientation of the screws, along with the various components, can be achieved along a desired axis. The base of the proximal portion 43, i.e. the area of connection with the distal portion 40 is truncated. The outer thread 42 is extended into it so as to achieve a safe anchoring.

The foregoing disclosure and description of the invention is illustrative and explanatory thereof. Various changes in the details of the illustrated construction can be made within the scope of the appended claims without departing from the true spirit of the invention. The present invention should only be limited by the following claims and their legal equivalents.

I claim:

1. A modular prosthetic acetabulum apparatus for use in reconstruction of a hip joint by placement in an acetabulum cavity, the apparatus comprising:
a supporting plate having a shape suitable for placement in the acetabulum cavity, said supporting plate having an annular internal surface, said annular internal surface tapering so as to have a wide diameter at an end of said supporting plate and a narrow diameter inwardly from said end of said supporting plate;
an anchoring means cooperative with said supporting plate for adjustably positioning said supporting plate in the acetabulum cavity to a desired depth;
a spacer locked onto said supporting plate, said spacer having an annular tapered interior, said annular tapered internal of said spacer having a wide diameter at a first end of said spacer, said annular tapered interior having a narrow diameter inwardly of said first end of said spacer, said spacer having an annular tapered external area formed adjacent a second end of said spacer, said annular tapered external area having a narrow diameter adjacent said second end of said spacer and a wide diameter inwardly from said second end, said annular tapered external area of said spacer being juxtaposed against an annular tapered surface of said supporting plate such that said spacer is selectively rotatable within said supporting plate; and
a cup affixed within said spacer, said cup having a tapered exterior surface received in said annular tapered interior of said spacer, said cup having an equatorial rim extending therearound and extending outwardly therefrom, said equatorial rim positioned against said first end of said spacer.

2. The apparatus of claim 1, said supporting plate having a plurality of external legs, at least one external leg of said plurality of legs having eyelets formed thereon, another of said plurality of external legs having an obturator hook extending therefrom, said anchoring means comprising:
a fastening screw of a spongy or cortical material received through one of said eyelets of the external leg.

3. The apparatus of claim 1, said supporting plate having at least one external leg and at least one internal leg, each of the external leg and the internal leg having eyelets formed therein, the eyelet having a frustoconical wall, said anchoring means comprising:
a fastening screw received through the eyelet, said fastening screw having a bearing surface with a hemispherical profile, said bearing surface bearing against said frustoconical wall of the eyelet.

4. The apparatus of claim 2, said plurality of external legs of said supporting plate comprising a pair of external legs having the eyelets formed therein, said plurality of external legs further comprising a single leg having said obturator hook formed thereon.

5. The apparatus of claim 4, said pair of external legs being disposed at an acute angle with respect to each other on one side of said supporting plate, said single leg with said obturator hook extending from an opposite side of said supporting plate.

6. The apparatus of claim 1, said tapered exterior surface of said cup being juxtaposed against said annular tapered interior of said spacer so as to be selectively rotatable within said annular tapered interior.

7. The apparatus of claim 1, said cup being formed of a metallic material.

8. The apparatus of claim 1, each of said supporting plate and said anchoring means and said cup having an osteoconductive coating thereon.

9. The apparatus of claim 1, said spacer having a locking collar extending from an exterior surface thereof.

10. The apparatus of claim 9, said locking collar extending around more that one-half of a circumference of said spacer.

11. The apparatus of claim 9, said locking collar having a plurality of eyelets formed in spaced relationship to each other along said locking collar, said supporting plate having a peripheral shoulder extending outwardly therefrom adjacent said end of said supporting plate, said peripheral shoulder having a plurality of tapped holes formed therein, the apparatus further comprising:
at least one connecting screw received in one of said plurality of eyelets and in one of said plurality of tapped holes so as to fix a rotation position of said spacer with respect to said supporting plate.

12. The apparatus of claim 11, each of said plurality of tapped holes having a wall with a frustoconical shape, the connecting screw having a head with a surface bearing against said wall.

13. The apparatus of claim 1, said cup having a smooth internal portion, the apparatus further comprising:
a polymeric insert received in said internal portion such that said polymeric insert moves freely therein, said insert having a hemispherical cavity suitable for receiving a head of a prosthetic femoral element.

14. The apparatus of claim 1, said cup having a smooth internal portion, said cup having a female portion, the apparatus further comprising:
a ceramic insert received in said female portion of said cup, said insert having a hemispherical cavity for receiving a head of a prosthetic femoral element.

15. The apparatus of claim 1, said anchoring means comprising a body having a cylindrically-shaped proximal portion and a distal portion with a sharp tip, said body having a spongy self-tapping outer thread, said proximal portion having a tapped channel formed axially therein, the apparatus further comprising:
a threaded piston received by said tapped channel, said threaded piston having a head and a tapped well, said tapped well suitable for receiving a locking counter-screw therein.

16. The apparatus of claim 15, said counter-screw having a head with a curved hemispherically-shaped bearing surface, the eyelet having a profile complementary to said bearing surface.
